(19) United States
(12) Patent Application Publication
(10) Pub. No.: US 2013/0216491 A1
(43) Pub. Date: Aug. 22, 2013

(54) COMPOSITION FOR CLEANING SCALP AND HEAD HAIR

(71) Applicants: Miyoko Ogihara, Osaka (JP); Momoko Shioyama, Osaka (JP); Masahiko Tanaka, Osaka (JP); Noboru Yoshino, Osaka (JP)

(72) Inventors: Miyoko Ogihara, Osaka (JP); Momoko Shioyama, Osaka (JP); Masahiko Tanaka, Osaka (JP); Noboru Yoshino, Osaka (JP)

(73) Assignee: OTSUKA PHARMACEUTICAL CO., LTD., Tokyo (JP)

(21) Appl. No.: 13/819,545

(22) PCT Filed: Aug. 9, 2011
(86) PCT No.: PCT/JP2011/068154

§ 371 Date: May 8, 2013

(30) Foreign Application Priority Data
Aug. 31, 2010 (JP) 2010-194281

(51) Int. Cl. A61K 8/44; A61Q 5/12; A61Q 5/02

(52) U.S. Cl. CPC A61K 8/44; A61Q 5/02; A61Q 5/12
USPC 424/70.13; 424/70.19; 424/70.17

(57) ABSTRACT

This invention provides a cleaning composition comprising a certain amount or more of an acylamino acid type surfactant, and further a non-ionic surfactant and a betaine-type ampholytic surfactant, which has a sufficient viscosity and exerts excellent conditioning effects.

### TECHNICAL FIELD

The present invention relates to a composition for cleaning (washing) scalp and head hair with an excellent feeling in use, which has a suitable viscosity and provides a conditioning benefit.

### BACKGROUND ART

In order to improve the conditioning performance of a hair cosmetic preparation such as a shampoo, various approaches have been done. As examples having such performance, hair cosmetic preparations comprising an anionic surfactant, a water-soluble resin, and a non-ionic surfactant and/or an ampholytic surfactant are known (see, Patent reference 1).

An acylamino acid type surfactant is a useful surfactant because it is less irritating and thereby it can clean skin and hair without damaging them, thus the acylamino acid type surfactant is used in a cleaning agent for body or hair (see, Patent reference 2).

Generally, a cleaning agent such as a body shampoo and a hair shampoo is required to have a certain level of viscosity because it is used after taking it out from a container thereof onto a palm. However, it is also known that the viscosity of a hair cleaning agent is reduced by using a high concentration of such acylamino acid type surfactant.

Hitherto, a thickener such as guar gum has been added thereto as a way to improve the low viscosity. But, it is known that some thickeners such as polysaccharides have problems such as color change (for example, white turbidity), and precipitation, if they are used.

It is known that an anionic surfactant is used in combination with a non-ionic surfactant in order to increase the viscosity of a cleaning agent while preventing said problem caused by the addition of thickeners such as color change and precipitation. However, it is difficult to increase the viscosity if an anionic surfactant of acylamino acid type is used as a main cleaning ingredient, because the addition of a non-ionic ingredient is unhelpful to increase the viscosity of such cleaning agent.

Due to the problem as mentioned above that a cleaning agent which contains an acylamino acid type surfactant as a main cleaning ingredient is not so easy to use because of the low viscosity, in fact, the use of an acylamino acid type surfactant is limited to a case of a low concentration of an acylamino acid type surfactant. As a result, the conditioning benefit provided by an acylamino acid type surfactant is not enough exerted despite its potential of conditioning benefit. Accordingly, it is tried to improve the conditioning performance by adding additional ingredients such as silicone, but silicone has a problem that it accumulates on hair/scalp and results in an oily feeling.

In addition, when an acylamino acid type surfactant is used in a cleaning composition in a high concentration, it has a disadvantage of reducing lather generation. There has not been known any cleaning composition comprising a high concentration of acylamino acid type surfactant which has a suitable viscosity for use and has an excellent quick-foaming property.

### [Patent Reference]

Patent reference 1: WO2005/074868
Patent reference 2: JP 2006-306908 A

### SUMMARY OF INVENTION

### Problems to be Solved by Invention

As described above, although an acylamino acid type surfactant is suitable for scalp/head hair application because it causes little irritation of skin, it is difficult to increase the viscosity of a hair formulation comprising an acylamino acid type surfactant compared with other commonly-used surfactants, and hence, it has been difficult to use a high concentration of an acylamino acid type surfactant in a cleaning agent such as a shampoo. The present invention provides a cleaning composition which comprises a high concentration of an acylamino acid type surfactant while keeping a suitable viscosity. It is also a purpose of the present invention to provide a cleaning composition comprising a high concentration of an acylamino acid type surfactant which additionally has an excellent conditioning performance.

### Means to Solve Problems

The present inventors have extensively studied to find out a solution of said problems, and then they have found that the use of a certain range of the amounts of a particular ampholytic surfactant and a non-ionic surfactant enables a cleaning composition comprising a high concentration of an acylamino acid type surfactant with a suitable viscosity, even without a thickener. They have completed a cleaning composition which is less irritating to scalp and additionally has an excellent protective effect on hair by using a cationic polymer in addition to the above-mentioned three ingredients. That is, the present invention provides a cleaning agent, especially those available as a shampoo or a conditioning shampoo, which provides users with a good usability and a good feeling in use while having a conditioning benefit even without silicone and the like.

Specifically, the present invention relates to a composition which comprises: 5 to 20% by weight of an acylamino acid type anionic surfactant, which is a higher amount compared to the usual case; a betaine-type ampholytic surfactant; and a non-ionic surfactant, and which has a high viscosity of 300 mPa·s or more. Moreover, the coacervate-forming ability of the cleaning composition is enhanced by adding a cationic polymer, and thereby the obtained cleaning composition has an excellent conditioning performance (for example, repairing damaged hair).

The present invention has been completed by further studying on the basis of these findings.

That is, the present invention provides the following aspects.

Item 1. A cleaning/washing composition which comprises

Ingredient (a): an acylamino acid type surfactant 5 to 20% by weight;

Ingredient (b): a betaine-type ampholytic surfactant; and

Ingredient (c): a non-ionic surfactant 1.5 to 10% by weight,

which has a viscosity ranging from 300 to 4000 mPa·s at 20° C.

Item 2. The cleaning/washing composition according to Item 1 wherein the acylamino acid type surfactant of Ingredient (a) is a compound represented by the formula:

R—NH—CH(COOX₁)((CH₂)ₙCOOX₂)

wherein

R is an acyl group having 8 to 20 carbon atoms;

n is 1 or 2;

X₁ and X₂ are each independently a hydrogen atom, an alkali metal, an alkaline earth metal, an ammonium group, or a cationic residue of a basic amino acid or an alkanolamine.

Item 3. The cleaning/washing composition according to Item 2, wherein R is selected from the group consisting of a capryloyl group, a caprinoyl group, a lauroyl group, a myristoyl group, a palmitoyl group, a stearoyl group, an oleoyl group, a cocoyl group (an acyl group derived from coconut oil fatty acid), an acyl group derived from palm kernel oil fatty acid, an acyl group derived from hydrogenated tallow fatty acid.
Item 4. The cleaning/washing composition according to any one of Items 1-3 wherein the acylamino acid type surfactant of Ingredient (a) is an anionic surfactant selected from the group consisting of lauroyl aspartic acid, cocoyl glutamic acid, and a salt thereof.
Item 5. The cleaning/washing composition according to any one of Items 1-4 which comprises 7 to 15% by weight of the acylamino acid type surfactant of Ingredient (a), and 2 to 5% by weight of the non-ionic surfactant of Ingredient (c).
Item 6. The cleaning/washing composition according to any one of Items 1-5 wherein the amount of Ingredient (b) is from 0.5 to 7 times by weight as much as the amount of Ingredient (c).
Item 7. The cleaning/washing composition according to any one of Items 1-6 wherein the non-ionic surfactant of Ingredient (c) is selected from the group consisting of a polyoxyalkylene-adduct type non-ionic surfactant, a polyoxypropylene.ethylene-adduct type non-ionic surfactant, an amine oxide type non-ionic surfactant, a mono- or di-ethanolamide type non-ionic surfactant, glycerol fatty acid esters, sorbitan fatty acid esters, sucrose fatty acid esters, a hydrogenated castor oil type non-ionic surfactant, an alkyl ether type non-ionic surfactant, and an alkyl glucoside type non-ionic surfactant.
Item 8. The cleaning/washing composition according to any one of Items 1-7 which further comprises Ingredient (d): a cationic polymer, wherein the amount of Ingredient (d) is from 0.025 to 1 times by weight as much as the amount of Ingredient (a).
Item 9. The cleaning/washing composition according to Item 8 wherein the cationic polymer of Ingredient (d) comprises cationic cellulose and/or dimethyldiallyl ammonium chloride.acrylamide copolymer.
Item 10. The cleaning/washing composition according to any one of Items 1-9 which further comprises

Ingredient (e): an anionic surfactant other than Ingredient (a) 0.1 to 5% by weight.

Item 11. The cleaning/washing composition according to Item 10 wherein the anionic surfactant of Ingredient (e) is selected from the group consisting of:

an anionic surfactant which has an acyl group having 6 to 20 carbon atoms and a taurine skeleton;

an anionic surfactant which has an acyl group having 6 to 20 carbon atoms and an alanine skeleton; and

an anionic surfactant which is a sulfonic acid compound having an alkyl group and/or an alkenyl group each of which has 10 to 20 carbon atoms, or a salt thereof.

Item 12. The cleaning/washing composition according to any one of Items 1-11 which is applied to scalp and head hair.
Item 13. A composition for cleaning/washing scalp and head hair which comprises

Ingredient (a): an acylamino acid type anionic surfactant which has an aspartate moiety or a glutamate moiety 5 to 20% by weight;

Ingredient (b): a betaine-type ampholytic surfactant;

Ingredient (c): a non-ionic surfactant; and

Ingredient (d): a cationic polymer,

wherein

the amount of Ingredient (b) is from 0.25 to 2 times by weight as much as the amount of Ingredient (a);

the amount of Ingredient (c) is from 0.1 to 1 times by weight as much as the amount of Ingredient (a); and

the amount of Ingredient (d) is from 0.025 to 1 times by weight as much as the amount of Ingredient (a).

Item 14. The composition for cleaning/washing scalp and head hair according to Item 13 wherein a coacervate is formed when the composition is diluted by 5 to 100-fold with water.
Item 15. The composition for cleaning/washing scalp and head hair according to Item 13 or 14 which further comprises

Ingredient (e): an anionic surfactant other than Ingredient (a).

Item 16. The composition for cleaning/washing scalp and head hair according to any one of Items 13-15 wherein the anionic surfactant of Ingredient (e) comprises

an anionic surfactant which has an acyl group having 6 to 20 carbon atoms and a taurine skeleton or an alanine skeleton; and/or

an anionic surfactant which is a sulfonic acid compound having an alkyl group and/or an alkenyl group each of which has 10 to 20 carbon atoms, or a salt thereof.

Item 17. The composition for cleaning/washing scalp and head hair according to any one of Items 13-16 wherein the Ingredient (e) is cocoyl methyl taurine, caproyl methyl taurine, lauroyl methyl alanine, tetradecenesulfonic acid, or a salt thereof.
Item 18. A cleaning/washing composition which comprises

Ingredient (a): an acylamino acid type anionic surfactant which has an aspartate moiety or a glutamate moiety 5 to 20% by weight;

Ingredient (b): a betaine-type ampholytic surfactant;

Ingredient (c): a non-ionic surfactant; and

Ingredient (e): an anionic surfactant which is selected from the group consisting of:

an anionic surfactant which has an acyl group having 6 to 20 carbon atoms and a taurine skeleton;

an anionic surfactant which has an acyl group having 6 to 20 carbon atoms and an alanine skeleton; and

an anionic surfactant which is a sulfonic acid compound having an alkyl group and/or an alkenyl group each of which has 10 to 20 carbon atoms, or a salt thereof, wherein

the amount of Ingredient (b) is from 0.25 to 2 times by weight as much as the amount of Ingredient (a);

the amount of Ingredient (c) is from 0.1 to 1 times by weight as much as the amount of Ingredient (a); and

the amount of Ingredient (e) is from 0.01 to 0.6 times by weight as much as the amount of Ingredient (a).

Item 19. The composition for cleaning/washing scalp and head hair according to Item 18 which further comprises

Ingredient (d): a cationic polymer

wherein the amount of Ingredient (d) is from 0.025 to 1 times by weight as much as the amount of Ingredient (a).

### Effects of the Invention

The cleaning (washing) composition of the present invention has a suitable viscosity though it comprises a high concentration of an acylamino acid type surfactant. The present invention can also provide a composition for cleaning scalp and head hair which has an excellent conditioning performance.

Specifically, the cleaning composition of the present invention comprises a betaine-type ampholytic surfactant and a non-ionic surfactant together with a given amount of an acylamino acid type surfactant, and thereby the present invention can provide a composition which has a suitable viscosity for use as a cleaning composition, causes little irritation of scalp, and provides a conditioning benefit. Moreover, the use of a cationic polymer in the composition enhances a coacervate formation, which may further improve smoothness of hair such as a smooth texture of hair in cleaning and a good smoothness in finger-combing after dried.

### BRIEF DESCRIPTION OF DRAWINGS

FIG. 1 is a graph as to Test example 5-1 showing a change of the coacervate formation in relation to the dilution of a washing liquid which comprises a cationic polymer besides an acylamino acid type surfactant, a betaine-type ampholytic surfactant and a non-ionic surfactant.

FIG. 2 is a graph as to Test example 5-2 showing a change of the coacervate formation in relation to the dilution of a washing liquid which comprises more than one kinds of cationic polymer besides an acylamino acid type surfactant, a betaine-type ampholytic surfactant, an non-ionic surfactant.

FIG. 3 depicts electron microscope photographs of hair surfaces as to Test example 7 which show a contrast between before and after treating damaged hair with Washing liquid 10.

### DESCRIPTION OF EMBODIMENTS

Next, embodiments of the present invention are shown below.

The cleaning composition of the present invention is characterized by comprising: a 5 to 20% by weight acylamino acid type surfactant; a betaine-type ampholytic surfactant; and a non-ionic surfactant.

The cleaning composition of the present invention includes, for example, what are used for cleaning scalp and/or head hair, or a whole body. Examples of the cleaning composition of the present invention include, in particular, a shampoo or a conditioning shampoo for scalp/head hair. In addition, the cleaning composition of the present invention may be used as a shampoo for a whole body.

The acylamino acid type surfactant used in the present invention is a surfactant having an acylamino acid moiety, and includes, for example, an anionic surfactant having an acylamino acid moiety.

In addition, the acylamino acid type surfactant used in the present invention may be a surfactant having two or more amino acid moieties wherein each of the amino acid moieties may be the same or different. The acylamino acid type surfactant used in the present invention may comprise two or more different acylamino acid type surfactants.

The acyl group of the acylamino acid type surfactant refers to a carbonyl group having a straight- or branched-chain saturated or unsaturated hydrocarbon group.

The acyl group includes, for example, acyl groups derived from caproic acid, caprylic acid, capric acid, lauric acid, myristic acid, palmitic acid, stearic acid, arachidic acid, palmitoleic acid, oleic acid, linoleic acid, linolenic acid, coconut oil fatty acid, castor oil fatty acid, olive oil fatty acid, palm oil fatty acid, palm kernel oil fatty acid, hydrogenated tallow fatty acid, and the like.

The number of carbon atoms of the acyl group is not limited, but an acyl group having 8 to 20 carbon atoms is preferable. From the viewpoint of an effect such as a smoothness in finger-combing after cleaning, an acyl group having 10 to 15 carbon atoms is preferable.

Examples of the acylamino acid type surfactant include lauroyl aspartic acid, cocoyl glutamic acid, myristoyl glutamic acid, coconut oil fatty acid acyl glutamic acid, lauroyl methyl alanine, cocoyl sarcosine, and cocoyl glycine, and a salt thereof.

Examples of the salt includes a metal salt such as sodium salt, potassium salt, lithium salt, magnesium salt, and calcium salt, an ammonium salt, an amine salt, a basic amino acid salt, a choline salt, and the like.

The preferred examples of an amino acid moiety comprised in an acylamino acid type surfactant include an acidic amino acid moiety such as aspartic acid and glutamic acid.

More preferred examples of the acylamino acid type surfactant include lauroyl aspartic acid, and cocoyl glutamic acid, and a salt thereof, which are available as AminoFoamer™ or Aminosurfact™ (Asahi Kasei Chemicals).

The most preferred examples of the anionic surfactant which has an acyl group having 10 to 15 carbon atoms and an acidic amino acid moiety used in the present invention include lauroyl aspartic acid, and cocoyl glutamic acid, and a salt thereof.

Preferred examples of the acylamino acid type surfactant include di-TEA-palmitoyl aspartate, diethyl palmitoyl aspartate, sodium lauroyl aspartate, zinc lauroyl aspartate, disodium capryloyl glutamate, cocoyl glutamic acid, disodium cocoyl glutamate, potassium cocoyl glutamate, sodium cocoyl glutamate, TEA-cocoyl glutamate, magnesium di-lauroylglutamate, sodium dilauramidoglutamide lysine, stearoyl glutamic acid, disodium stearoyl glutamate, aluminum stearoyl glutamate, potassium stearoyl glutamate, sodium stearoyl glutamate, dioctyldodecyl stearoyl glutamate, magnesium palmitoyl glutamate, myristoyl glutamic acid, potassium myristoyl glutamate, sodium myristoyl glutamate, lauroyl glutamic acid, disodium lauroyl glutamate, potassium lauroyl glutamate, sodium lauroyl glutamate, and TEA-lauroyl glutamate.

The acylamino acid type surfactant used in the present invention may comprise two or more different acylamino acid type surfactants.

Preferred examples of the acylamino acid type surfactant include a compound represented by the formula:

R—NH—CH(COOX₁)((CH₂)ₙCOOX₂)

wherein

R is an acyl group having 8 to 20 carbon atoms, preferably having 10 to 15 carbon atoms;

n is 1 or 2;

X₁ and X₂ are each independently a hydrogen atom, an alkali metal (for example, sodium or potassium), an alkaline earth metal (for example, calcium or magnesium), an ammonium group, or a cationic residue of a basic amino acid (for example, lysine or arginine) or an alkanolamine (for example, diethanolamine or triethanolamine).

Preferred examples of the above R include a capryloyl group, a caprinoyl group, a lauroyl group, a myristoyl group, a palmitoyl group, a stearoyl group, an oleoyl group, a cocoyl group, an acyl group derived from palm kernel oil fatty acid, and an acyl group derived from hydrogenated tallow fatty acid.

Examples of the above R having 10 to 15 carbon atoms include a caprinoyl group, a lauroyl group, a myristoyl group, and a cocoyl group.

The cleaning composition of the present invention may comprise an acylamino acid type surfactant in the range of from 5 to 20% by weight, preferably from 7 to 15% by weight, and more preferably from 7.5 to 12% by weight.

The ampholytic surfactant used in the present invention refers to a surfactant having both of a cation (cation group) an anion (anion group).

Preferred examples of the ampholytic surfactant used in the present invention include an ampholytic surfactant having a betaine skeleton which may be substituted partially (hereinafter referred to as “betaine-type ampholytic surfactant”).

Examples of the betaine-type ampholytic surfactant include:

a carboxy betaine type including:

alkyl carboxy betaine such as lauryl dimethyl betaine, myristyl dimethyl betaine, palmityl dimethyl betaine, stearyl dimethyl betaine, oleyl dimethyl betaine, coconut alkyl dimethyl betaine, lauryl methyl ethyl betaine, lauryl dihydroxyethyl betaine, stearyl dihydroxyethyl betaine, coconut alkyl dihydroxyethyl betaine, and the like;

ether carboxy betaine such as octadecyloxy methyl dimethyl betaine and the like; and

amide carboxy betaine such as lauramidopropyl dimethyl betaine, myristamidopropyl dimethyl betaine, stearamidopropyl dimethyl betaine, oleamidopropyl dimethyl betaine, cocamidopropyl dimethyl betaine, lauramidopropyl betaine, and the like;

a sulfobetaine type including:

alkyl sulfobetaine such as lauryl dimethyl hydroxypropyl sulfobetaine, myristyl dimethyl hydroxypropyl sulfobetaine, lauryl dimethyl propyl sulfobetaine, coconut alkyl dimethyl propyl sulfobetaine, and the like; and

amide sulfobetaine such as lauramidopropyl dimethyl hydroxypropyl sulfobetaine, lauramidopropyl hydroxysultaine, and the like;

an imidazoline type including:

lauryl carboxymethyl hydroxyethyl imidazolinium betaine, myristyl carboxymethyl hydroxyethyl imidazolinium betaine, stearyl carboxymethyl hydroxyethyl imidazolinium betaine, alkyl carboxymethyl hydroxyethyl imidazolinium betaine, and the like,

a glycine type, an aminopropionic acid type, a sulfonic acid type, a sulfuric acid type, an aminocarboxylate type, a phosphoric acid type or lecithin, and the like.

Preferred examples of the betaine-type surfactant used in the present invention include a betaine-type surfactant which has a betaine skeleton and a fatty acid moiety having 10 to 18 carbon atoms. If the fatty acid moiety has less than 10 carbon atoms, such betaine-type surfactant may cause an irritation. If the fatty acid moiety has more than 18 carbon atoms, such betaine-type surfactant may reduce lathering.

The betaine-type ampholytic surfactant used in the present invention may comprise two or more different betaine-type ampholytic surfactants.

The cleaning composition of the present invention may comprise a betaine-type ampholytic surfactant in the range of from 1 to 20% by weight, preferably from 2 to 15% by weight, and more preferably from 3 to 10% by weight.

The non-ionic surfactant used in the present invention may be any non-ionic surfactant which is usually available for use in cosmetics and the like, and includes, for example, polyoxyethylene (POE) alkyl ether, POE polyoxypropylene glycol, POE aryl ether, POE hydrogenated castor oil ether, POE castor oil ether, POE lanolin alcohol ether, POE glycerin fatty acid ester (for example, POE glyceryl monococoate), POE sorbitan fatty acid ester (for example, POE sorbitan triisostearate), POE sorbitol fatty acid ester, polyethylene glycol fatty acid ester, glycerol fatty acid ester, polyglycerol fatty acid ester, sorbitan fatty acid ester, ethylene glycol fatty acid ester, propylene glycol fatty acid ester, butylene glycol fatty acid ester (for example, butylene glycol laurate), pentaerythritol fatty acid ester, a sugar derivative such as alkylated polysaccharide etc., alkyl glyceryl ether, organic acid monoglyceride, fatty acid alkanolamide (for example, cocamide methy MEA), POE alkyl amine, amine oxide, and the like.

In addition, examples of the non-ionic surfactant used in the present invention include an ether type non-ionic surfactant which is in the form of a condensation product of alkyl glycol and propylene glycol, and includes, for example, lauryl glycol hydroxypropyl ether or myristyl glycol hydroxypropyl ether.

Moreover, examples of the non-ionic surfactant used in the present invention include a polyoxyalkylene-adduct type non-ionic surfactant (for example, butylene glycol laurate), a polyoxypropylene ethylene-adduct type non-ionic surfactant, an amine oxide type non-ionic surfactant, a mono- or di-ethanolamide type non-ionic surfactant (for example, cocamide methy MEA), glycerol fatty acid esters (for example, POE glyceryl monococoate), sorbitan fatty acid esters (for example, POE sorbitan triisostearate), sucrose fatty acid esters, a hydrogenated castor oil type non-ionic surfactant, an alkyl ether type non-ionic surfactant (for example, lauryl glycol hydroxypropyl ether), and an alkyl glucoside type non-ionic surfactant.

The non-ionic surfactant used in the present invention may comprise two or more different non-ionic surfactants.

The cleaning composition of the present invention may comprise a non-ionic surfactant in the range of from 1.5 to 10% by weight, preferably from 2 to 5% by weight.

It is preferable that the composition of the present invention has a suitable viscosity for cleaning. The composition of the present invention preferably has a viscosity ranging from 300 to 4000 mPa·s, more preferably, ranging from 400 to 3000 mPa·s. The composition of the present invention having such viscosity may be in a suitable state for using as a body shampoo and a shampoo which are used after taking it out from a container thereof onto a palm.

A method to determine a viscosity is not limited, but includes, for example, a method using a B type viscometer with a rotor (No. 2 or No. 3) at a rate of 12 or 30 rpm at 20° C. Other examples of the method to determine a viscosity include ways specified in Japanese Pharmacopoeia (General Tests, Viscosity Determination; Method II viscosity measurement by rotational viscometer, Single cylinder-type rotational viscometer, 20° C., 12 or 30 rpm, Rotor No. 2 or No. 3).

In the cleaning composition of the present invention, the amount of a betaine-type ampholytic surfactant may be from 0.25 to 2 times, preferably from 0.3 to 1.5 times, more preferably 0.4 to 1 times, by weight as much as the amount of an acylamino acid type surfactant.

In the cleaning composition of the present invention, the amount of a non-ionic surfactant may be preferably from 0.1 to 1 times, more preferably from 0.15 to 0.7 times, further more preferably from 0.2 to 0.5 times, by weight as much as the amount of an acylamino acid type surfactant.

In the cleaning composition of the present invention, the amount of a betaine-type ampholytic surfactant may be preferably from 0.5 to 7 times, more preferably from 1 to 5 times, further more preferably from 1.5 to 3 times, by weight as much as the amount of a non-ionic surfactant, in order to give the above mentioned viscosity.

The coacervate-forming ability of the composition of the present invention may be enhanced by adding Ingredient (d): a cationic polymer compound, and thereby the obtained composition may have an improved hair conditioning performance and may improve the feeling after cleaning.

Examples of the cationic polymer used in the present invention include any cationic polymer having a cationic moiety, and include, but not limited to, for example, cationic cellulose such as O-[2-hydroxy-3-(trimethylammonio)propyl]hydroxyethylcellulose chloride, [hydroxy(trimethylammonio)propyl]hydroxyethylcellulose chloride, and the like, a cationic hydrolyzed protein, a cationic vinyl type or acrylic type polymer (for example, dimethyldiallyl ammonium chloride.acrylamide copolymer), and a mixture thereof. Cationic cellulose, and a cationic vinyl type or acrylic type polymer are preferable.

Other examples of the cationic polymer include, for example, polyquaternium-4/hydroxypropyl starch copolymer, polyquaternium-10, polyquaternium-11, polyquaternium-64, polyquaternium-7, and the like.

The cationic polymer used in the present invention may comprise two or more different cationic polymers.

In the cleaning composition of the present invention, preferably, the amount of a cationic polymer is adjusted according to the amount of an acylamino acid type surfactant from the viewpoints of the coacervate-forming ability, and the amount of a cationic polymer may be from 0.025 to 1 times, preferably 0.03 to 0.6 times, more preferably 0.04 to 0.5 times, by weight as much as the amount of an acylamino acid type surfactant.

The coacervate (or coacervation) as used herein is an insoluble complex which is formed by diluting a washing liquid such as shampoo which comprises a surfactant and a cationic polymer with water to lay the concentration in a particular range. The formed coacervate plays a role in a hair conditioning performance. For example, it is possible to reduce a friction between hair and fingers, and thereby it becomes possible to provide a good hair texture and a good feeling in finger-combing as well as an improved smoothness in finger-combing or combing after cleaning. The coacervate can remain after hair cleaning, especially even after rinsing and thereby it can bring in a hair conditioning benefit after cleaning.

The cleaning composition of the present invention may provide such coacervate benefits in various use situation, because the coacervate benefit is provided in cleaning scalp/head hair (the relative concentration of the cleaning composition during cleaning per its neat cleaning composition is usually in the range of 0.1 to 0.2), and in rinsing it (the relative concentration of the cleaning composition during rinsing per its neat cleaning composition is usually 0.1 or less).

The cleaning composition of the present invention may have pH which is usually available for scalp/head hair, however, it is preferable that the pH is in a particular range in order to improve a coacervate formation. The cleaning composition has preferably the pH being in the range of 4 to 8. In order to stabilize the viscosity of the cleaning composition, the cleaning composition more preferably has the pH being in the range of 4 to 6.5.

In addition, the composition of the present invention may further comprise Ingredient (e): an anionic surfactant in order to improve the quick-foaming property of an acylamino acid type surfactant. Preferably, said anionic surfactant is an anionic surfactant other than an acylamino acid type (Ingredient (a)) which has an amino acid moiety such as an aspartate moiety, a glutamate moiety and the like. Examples of the anionic surfactant include lauroyl methyl taurine, lauroyl methyl alanine (lauroyl methyl beta-alanine), tetradecene sulfonic acid, cocoyl methyl taurine, cocoyl ethyl ester sulfonic acid, caproyl methyl taurine, and a salt thereof (for example, sodium salt, potassium salt, magnesium salt, or triethanolamine salt).

Furthermore, examples of Ingredient (e): an anionic surfactant include:

an anionic surfactant which has an acyl group (preferably having 6 to 20 carbon atoms) and a taurine skeleton or an alanine skeleton, such as cocoyl methyl taurine, caproyl methyl taurine, lauroyl methyl alanine (lauroyl methyl beta-alanine) and a salt thereof, and

an anionic surfactant which is a sulfonic acid compound having an alkyl group and/or an alkenyl group each of which has 10 to 20 carbon atoms, and a salt thereof, such as alkylbenzene sulfonic acid, tetradecene sulfonic acid, and a salt thereof. The anionic surfactant used in the present invention may comprise two or more different anionic surfactants.

Examples of the anionic surfactant which has an acyl group and a taurine skeleton include a compound represented by the formula:

R—N(CH₃)((CH₂)₂SO₃X₁)

wherein

R is an acyl group having 6 to 20 carbon atoms,

X₁ is a hydrogen atom, an alkali metal (for example, sodium or potassium), an alkaline earth metal (for example, calcium or magnesium), an ammonium group, or a cationic residue of a basic amino acid (for example, lysine or arginine) or an alkanolamine (for example, diethanolamine or triethanolamine), and

the specific examples thereof include cocoyl methyl taurine and caproyl methyl taurine.

Said acyl group is the same as defined as to the acylamino acid type surfactant, and includes, for example, caproyl group, capryloyl group, caprinoyl group, lauroyl group, myristoyl group, palmitoyl group, stearoyl group, oleoyl group, cocoyl group, an acyl group derived from palm kernel oil fatty acid, and an acyl group derived from hydrogenated tallow fatty acid.

Examples of the anionic surfactant which has an acyl group and an alanine skeleton include a compound represented by the formula:

R—N(CH₃)((CH₂)₂COOX₁)

wherein

R is an acyl group having 6 to 20 carbon atoms,

X₁ is a hydrogen atom, an alkali metal (for example, sodium or potassium), an alkaline earth metal (for example, calcium or magnesium), an ammonium group, or a cationic residue of a basic amino acid (for example, lysine or arginine) or an alkanolamine (for example, diethanolamine or triethanolamine), and

the specific examples thereof include lauroyl methyl alanine (lauroyl methyl beta-alanine).

Said acyl group is the same as defined as to the acylamino acid type surfactant, and includes, for example, caproyl group, capryloyl group, caprinoyl group, lauroyl group, myristoyl group, palmitoyl group, stearoyl group, oleoyl group, cocoyl group, an acyl group derived from palm kernel oil fatty acid, and an acyl group derived from hydrogenated tallow fatty acid.

In the present invention, examples of the sulfonic acid compound having an alkyl group and/or an alkenyl group each of which has 10 to 20 carbon atoms include alkyl(C₁₀₋₁₄) benzene sulfonic acid, α-olefin(C₁₀₋₂₀) sulfonic acid, α-olefin(C₁₂₋₁₆) sulfonic acid, α-olefin(C₂₋₁₄) sulfonic acid, and α-olefin(C₁₄₋₁₆) sulfonic acid.

The composition of the present invention may comprise an anionic surfactant which is added in order to improve the quick-foaming property in the range of from 0.1 to 5% by weight, preferably from 1 to 4% by weight.

In the cleaning composition of the present invention, preferably, the amount of an anionic surfactant of Ingredient (e) which is added in order to improve the quick-foaming property may be from 0.01 to 0.6 times, more preferably from 0.1 to 0.5 times, by weight as much as the amount of an acylamino acid type surfactant of Ingredient (a).

The composition of the present invention may comprise an algefacient-ingredient in order to enhance a refreshing feeling without increasing an irritation. Said algefacient-ingredient may be a naturally-derived compound, a synthetic compound, and a salt thereof, and also the algefacient-ingredient may be any formation comprising these compounds. In addition, an algefacient-ingredient may be one algefacient-ingredient, or may be a combination of two or more algefacient-ingredients.

Preferred examples of the algefacient-ingredient which may be used in the composition of the present invention include menthol, and an essential oil, a plant-derived ingredient and the like which are capable of providing a cooling feeling.

The cleaning composition of the present invention may comprise an algefacient-ingredient preferably in the range of 0.1% by weight or more, more preferably from 0.2 to 0.6% by weight. If the amount of an algefacient-ingredient is more than 0.6% by weight, the cleaning composition may cause an irritation of skin, and thereby the advantage of an acylamino acid type surfactant may be impaired.

The silicone-ingredient as described herein includes, for example, a polymer which has silicon oxide (SiO) as a basic skeleton and may have a hydrophilic substituent group. Examples of a silicone-ingredient include, for example, methylphenyl polysiloxane, methyl polysiloxane, octamethylcyclotetrasiloxane, decamethylcyclopentasiloxane, dodecamethylcyclohexasiloxane, methylcyclopolysiloxane, octamethyltrisiloxane, decamethyltetrasiloxane, polyoxyethylene methylpolysiloxane copolymer, polyoxypropylene methylpolysiloxane copolymer, poly(oxyethylene/oxypropylene) methylpolysiloxane copolymer, methylhydrogenpolysiloxane, tetrahydrotetramethylcyclotetrasiloxane, stearoxymethylpolysiloxane, cetoxymethylpolysiloxane, methylpolysiloxane emulsion, highly polymerized methyl polysiloxane, trimethylsiloxysilicate, crosslinked methylpolysiloxane, crosslinked methylphenylpolysiloxane, and the like. Examples of the silicone-ingredient as described herein also include various derivatives such as an amino-modified silicone, an epoxy-modified silicone, a carboxyl-modified silicone, a carbinol-modified silicone, a methacryl-modified silicone, a mercapto-modified silicone, a phenol-modified silicone, a single-end reactive silicone, a co-modified silicone, a polyether-modified silicone, a methylastyryl-modified silicone, a alkyl-modified silicone, a higher fatty acid ester-modified silicone, a special hydrophilically-modified silicone, a higher alkoxy-modified silicone, a higher fatty acids-containing silicone, a fluorine-modified silicone and the like.

In the present invention, “a silicone-ingredient is not comprised (without a silicone-ingredient)” means that a silicone-ingredient other than a left one due to a carryover or a left one in a storage solution is not comprised substantively. In addition, the “an effective amount of a silicone-ingredient is not comprised” also means that the concentration of a silicone-ingredient is not enough to show a conditioning benefit such as hair coating benefit and the like.

The composition of the present invention may have the high transparency, because the viscosity of the composition may be increased even without a thickener (for example, cellulose gum, guar gum, gellan gum, sclerotium gum, cellulose gum, and the like, guar gum) and the like.

In order to prevent color change of the composition of the present invention due to oxidation during a long storage and in order to keep the composition transparent, the composition of the present invention may comprise an antioxidant such as sodium sulfite etc. and a color inhibitor at a concentration of, for example, from 0.01 to 0.5% by weight. When the composition of the present invention has the high transparency, it is easy to color the composition, and the color of the composition may be changed to the desired color by adding a commercially available coloring agent.

The composition of the present invention may comprise a solvent which is available for a cleaning composition. Preferred examples of the solvent include water, dipropylene glycol, and the like.

Preferably, the concentration of dipropylene glycol lies in the range of from 0.5 to 5% by weight.

When the composition of the present invention comprises water, a suitable amount of water may be used according to amounts of the other ingredients.

The composition of the present invention may comprise other ingredients which are usually used in a cleaning composition, besides the above ingredients. Examples of “ingredients which are usually used in a cleaning composition” as used herein include a surfactant, a polymer ingredient, a moisturizing agent, a preservative agent, a bactericidal agent such as isopropyl methylphenol and the like, an anti-inflammatory agent such as glycyrrhizinic acid/glycyrrhizinate and the like, an pH adjuster such as sodium hydroxide, citric acid, and the like, a sequestering agent such as EDTA and the like, a tonicity agent, a flavor, and the like, in addition to the above-mentioned ingredients. In addition, the composition of the present invention may further comprise a conditioner-ingredient capable of protecting a surface of hair.

Moreover, the cleaning composition of the present invention may optionally include an active ingredient. The active ingredient as used herein refers to an ingredient which may provide scalp/head hair with a beneficial effect. Examples of the active ingredient include, but not limited to, a hair growing agent, a hair regrowth agent, a deodorant, an anti-dandruff agent and the like. Examples of the active ingredient include, for example, adenosine, adenosine phosphate, vitamins, minerals, edetic acid, milk-derived ingredients such as whey and the like, carpronium chloride, matricaria oil, hinokitiol, benzylaminopurine, minoxidil, finasteride and a salt or derivative thereof, ginger, ginkgo, aloe, garlic, glycyrrhiza, chamomile, fennel, tincture, Swertia Herb, mulberry root bark, ginseng, moutan bark, Chinese Caterpillar Fungus, seaweed-derived ingredients such as fucoidan and the like, Chitofilmer, salicylic acid, inositol, and the like.

### EXAMPLES

The following Examples and Test examples serve to illustrate the present invention in more detail, which should not intend to limit the present invention.

Unless otherwise specified, each amount of ingredients in the Examples is represented by % by weight (w/w %).

### Test Example 1

### Test as to Change in the Feeling in Use, in Relation to the Concentration of Acylamino Acid Type Surfactant

In order to evaluate the change in the feeling in use of a formulation for cleaning head hair in relation to the difference of acylamino acid type surfactants, the amount thereof, and a combination use thereof, cleaning formulations as showed in Table 1 were prepared, and then the sensory evaluation was done.

### (Test Method and Result)

Cleaning formulations as shown in Table 1 were prepared, and an appropriate amount of each cleaning formulation was applied onto the previously-wetted head hair and foamed. The feelings in use during and after cleaning head hair were evaluated (the amounts in the table are represented by a percentage by weight).

TABLE 1

| | Control | Control | Example | Example | Example |
|---|---|---|---|---|---|
| | 1 | 2 | 1 | 2 | 3 |
| Sodium Lauroyl Aspartate *1 | 3.8 | 3.8 | 7.5 | 8 | 8 |
| Sodium Cocoyl Glutamate *2 | | | | 0.9 | 0.9 |
| Sodium Methyl Cocoyl Taurate *3 | 3 | 3 | | 3.6 | 3.6 |
| Sodium Cocoyl Ethyl Ester | | | | 0.5 | 0.5 |
| Sulfonate *4 | | | | | |
| Sodium Tetradecenesulfonate *5 | 2 | 2 | 2 | | |
| Alkyl Carboxymethyl Hydroxyethyl | 6 | 6 | 4.5 | 2.1 | 6 |
| Imidazolinium Betaine *6 | | | | | |
| Lauramidopropyl Hydroxysultaine *7 | | | | 4 | |
| Lauryl Glycol Hydroxypropyl Ether *8 | | 2 | | 2 | 2 |
| POE Sorbitan Triisostearate *9 | | 1 | | 2 | 2 |
| Cationic Polymer | 0.6 | 1.1 | 0.6 | 0.8 | 0.8 |
| Moisturizing Agent• | Suitable | Suitable | Suitable | Suitable | Suitable |
| Preservative Agent• | Amount | Amount | Amount | Amount | Amount |
| Bactericidal Agent• | | | | | |
| pH Adjuster etc. | | | | | |
| Purified Water | Balance | Balance | Balance | Balance | Balance |
| Total | 100 | 100 | 100 | 100 | 100 |
| Feeling in use | Δ | Δ | ◯ | ◯ | ◯ |

*1: Aminofoamer FLDS-L (25% Sodium Lauroyl Aspartate-containing fluid (Asahi Kasei Chemicals))
*2: Aminosurfact ACMT-L (30% Sodium Cocoyl Glutamate-containing fluid (Asahi Kasei Chemicals))
*3: DIAPON K-SF (30% Sodium Methyl Cocoyl Taurate-containing fluid (NOF Corporation))
*4: Jordapon CI P (BASF Japan Ltd.)
*5: Lipolan LJ-441 (about 35% Sodium Tetradecenesulfonate-containing fluid (LION Corporation))
*6: Softazoline CH-R (30% Alkyl Carboxymethyl Hydroxyethyl Imidazolinium Betaine-containing fluid (Kawaken Fine Chemicals))
*7: Softazoline LSB-R (about 30% Lauramidopropyl Hydroxysultaine-containing fluid (Kawaken Fine Chemicals))
*8: Viscosafe LPE (Kawaken Fine Chemicals)
*9: Rheodol TW-IS399c (Kao Corporation)

The feelings in use showed in Table 1 were evaluated on the basis of the irritation of scalp in cleaning head hair, the smoothness in finger-combing in rinsing away, and the dry feeling of scalp/head hair after use.

Results are showed as follows:

Feeling in use was unsatisfying: Δ;

Feeling in use was satisfying: ∘.

The cleaning formulations were prepared by a conventional procedure.

The results showed that the use of 5% by weight or more of an acylamino acid type surfactant having an acidic amino acid moiety is preferable to improve the feeling in use, in particular, an amino acid such as aspartic acid and glutamic acid is more preferable.

### Test Example 2

### Test of Viscosity

In order to improve the low viscosity of a cleaning formulation comprising an acylamino acid type surfactant, the viscosity change in the combination of an acylamino acid type surfactant, an ampholytic surfactant and/or a non-ionic surfactant was examined.

Method to Determine Viscosity:

The viscosity of the composition of the present invention was determined with a B type viscometer (BL type (TOKI SANGYO)). Rotor No. 2 was used for a composition with a low viscosity (<400 mPa·s) and rotor No. 3 was used for a composition with a relatively high viscosity. The viscosity was determined at 20° C. at a rate of 12 or 30 rpm according to a conventional way.

Sodium lauroyl aspartate and triethanolamine-cocoyl glutamate were mixed in equal amount to give a mixture as an acylamino acid type surfactant. Then, in order to find a suitable combination of an ampholytic surfactant and a non-ionic surfactant for improving the low viscosity of an acylamino acid type surfactant, the amino acid type surfactant mixture, a non-ionic surfactant, and an ampholytic surfactant were mixed, wherein the final concentration of a non-ionic surfactant was 3% by weight, and the final concentration of a combination of the amino acid type surfactant mixture and an ampholytic surfactant was 15% by weight. Water was added to the resulting mixture to give a composition comprising three different surfactants (i.e., said composition was a mixture of the acylamino acid type surfactant mixture, a non-ionic surfactant, an ampholytic surfactant and water), and then the viscosity changes thereof were examined.

Table 2 indicates ampholytic surfactants and non-ionic surfactants used herein. As an ampholytic surfactant, lauramidopropyl betaine, lauramidopropyl hydroxysultaine, alkyl carboxymethyl hydroxyethyl imidazolinium betaine, and laurylaminodiacetate are used. As a non-ionic surfactant, butylene glycol laurate, lauryl glycol hydroxypropyl ether, and cocamide methy MEA are used.

The term “the viscosity change” used herein means the increase/decrease in the viscosity of a composition comprising three different surfactants (i.e., said composition is a mixture of the acylamino acid type surfactant mixture, a non-ionic surfactant, an ampholytic surfactant and water) compared with the viscosity of a composition comprising the acylamino acid type surfactant mixture (i.e., said composition is a mixture of 15% by weight of the acylamino acid type surfactant mixture and water).

The compositions tested as to the viscosity comprised 3% by weight of a non-ionic surfactant (butylene glycol laurate, lauryl glycol hydroxypropyl ether, or cocamide methy MEA), and 15% by weight of a combination of the amino acid type surfactant mixture and an ampholytic surfactant.

The results are shown in Table 2.

TABLE 2

| | | Butylene | Lauryl Glycol | Cocamide |
|---|---|---|---|---|
| | | Glycol | Hydroxypropyl | Methy |
| | Content * | Laurate *5 | Ether *6 | MEA *7 |
| Lauramidopropyl | 6-9% | + | + | − |
| Betaine *1 | 9-12% | ++ | ++ | + |
| Lauramidopropyl | 6-9% | − | − | − |
| Hydroxysultaine *2 | 9-12% | +++ | +++ | + |
| Alkyl | 6-9% | − | + | − |
| Carboxymethyl | | | | |
| Hydroxyethyl | 9-12% | + | ++ | ++ |
| Imidazolinium | | | | |
| Betaine *3 | | | | |
| Laurylaminodi- | 6-9% | − | − | − |
| acetate *4 | 9-12% | − | − | − |

* “Content” means the amount (% by weight) of an ampholytic surfactant in a composition. The total amount of the mixture of acylamino acid type surfactants and an ampholytic surfactant is 15% by weight of a composition.
*1: Amphorex LB-2 (about 30% by weight Lauramidopropyl Betaine-containing fluid (MIYOSHI OIL & FAT CO., LTD.))
*2: Softazoline LSB-R (about 30% by weight Lauramidopropyl Hydroxysultaine-containing fluid (Kawaken Fine Chemicals))
*3: Lebon CIB (about 35% by weight Alkyl Carboxymethyl Hydroxyethyl Imidazolinium Betaine-containing fluid (Sanyo Chemical Industries, Ltd.))
*4: Nissan Anon LA (about 30% by weight Laurylaminodiacetate-containing fluid (NOF CORPORATION))
*5: Compol BL (NOF CORPORATION)
*6: Viscosafe LPE (Kawaken Fine Chemicals)
*7: Aminon C-11S (Kao Corporation)
In Table 2:
the symbol “−” denotes no change of the viscosity;
the symbol “+” denotes an increase in the viscosity (to 200 mPa · s);
the symbol “++” denotes a large increase in the viscosity (200 to 1000 mPa · s);
the symbol “+++” denotes a very large increase in the viscosity (>1000 mPa · s)

The results showed that the low viscosity of a composition comprising an acylamino acid type surfactant is improved by adding an ampholytic surfactant having a betaine skeleton besides an acylamino acid type surfactant and a non-ionic surfactant, and thereby the viscosity of the composition was increased.

Additionally, the compositions whose viscosity was enhanced in the above viscosity test also showed an improved quality of foam, an improved durability of foam, and an improved feeling in use, when the compositions were used in cleaning.

### Test Example 3

### Viscosity Change in Relation to the Concentration of Non-Ionic Surfactant

In order to evaluate the viscosity change of a composition comprising an acylamino acid type surfactant in relation to the amount of a non-ionic surfactant, Test compositions 1 to 4 as shown in table 3 were prepared, and the viscosity changes were examined. POE glyceryl monococoate was used as a non-ionic surfactant (the amounts in the table are represented by a percentage by weight).

TABLE 3

| | Test | Test | Test | Test |
|---|---|---|---|---|
| | Composi- | Composi- | Composi- | Composi- |
| | tion 1 | tion 2 | tion 3 | tion 4 |
| Sodium Lauroyl | 8.5 | 8.5 | 8.5 | 8.5 |
| Aspartate | | | | |
| Lauramidopropyl | 4.5 | 4.5 | 4.5 | 4.5 |
| Betaine *1 | | | | |
| Sodium Tetradecene- | 2 | 2 | 2 | 2 |
| sulfonate | | | | |
| POE Glyceryl | 0 | 2 | 3 | 5 |
| Monococoate *2 | | | | |
| Cationic Polymer | 3 | 3 | 3 | 3 |
| Bactericidal Agent• | Suitable | Suitable | Suitable | Suitable |
| Preservative Agent• | Amount | Amount | Amount | Amount |
| Anti-Inflammatory | | | | |
| Agent• | | | | |
| Moisturizing Agent | | | | |
| Solvent• | Suitable | Suitable | Suitable | Suitable |
| pH Adjuster• | Amount | Amount | Amount | Amount |
| Flavor• | | | | |
| Algefacient | | | | |
| Purified Water | Balance | Balance | Balance | Balance |
| Total | 100 | 100 | 100 | 100 |
| Viscosity (mPa · s) | 125 | 575 | 600 | 725 |

*1: Nissan Anon BDL-SF (30% Lauramidopropyl Betaine-containing fluid (NOF Corporation))
*2: Glycerox HE (Croda Japan KK)

The viscosity was determined according to the method of Test example 2. The results showed that a non-ionic surfactant is necessary to increase the viscosity of a composition. The results showed that Test compositions 2-4 had a viscosity of 300 mPa·s or more, and hence they have a suitable viscosity for using as an shampoo and the like.

### Test Example 4

### Improvement of Quick-Foaming Property

As for compositions tested in Test examples 1 to 3, the change in the lather of a composition comprising an anionic surfactant was examined in order to improve the lather, especially improve the quick-foaming property.

In order to measure the volume of a quickly-occurring foam, a control composition and Test compositions 1-5 were prepared as shown in Table 4 (the amounts in the table are represented by a percentage by weight). Then, each of the control composition and Test compositions 1-5 was diluted by 7-fold with purified water, and the resulting dilutions were stirred with fiberMixer (MX-X58 (Panasonic)) at a slow speed mode at room temperature for 5 seconds. The foam volume (mL) obtained shortly after stirring was treated as a volume of a quickly-occurring foam, and the differences between the foam volumes of the control composition and the test compositions were evaluated.

TABLE 4

| | Control | Test | Test | Test | Test | Test |
|---|---|---|---|---|---|---|
| | Composition | Composition 1 | Composition 2 | Composition 3 | Composition 4 | Composition 5 |
| Sodium Lauroyl | 8 | 10 | 8 | 8 | 8 | 8 |
| Aspartate | | | | | | |
| Alkyl Carboxymethyl | 3 | 3 | 3 | 3 | 3 | 3 |
| Hydroxyethyl | | | | | | |
| Imidazolinium Betaine | | | | | | |
| POE Glyceryl | 2 | 2 | 2 | 2 | 2 | 2 |
| Monococoate | | | | | | |
| Sodium Cocoyl | | | 2 | | | |
| Glutamate | | | | | | |
| Sodium Methyl Cocoyl | | | | 2 | | |
| Taurate | | | | | | |
| Sodium Lauroyl | | | | | 2 | |
| Methylaminopropionate *1 | | | | | | |
| Sodium | | | | | | 2 |
| Tetradecenesulfonate | | | | | | |
| pH adjuster | Suitable | Suitable | Suitable | Suitable | Suitable | Suitable |
| | Amount | Amount | Amount | Amount | Amount | Amount |
| Purified Water | Balance | Balance | Balance | Balance | Balance | Balance |
| Total | 100 | 100 | 100 | 100 | 100 | 100 |
| pH | 6.5 | 6.5 | 6.5 | 6.5 | 6.5 | 6.5 |
| Foam Volume (mL) | 533 | 562 | 557 | 593 | 593 | 630 |
| Δ value | 0 | 28 | 23 | 60 | 60 | 97 |
| (Test composition | | | | | | |
| control composition) | | | | | | |

*1: Alanon ALE (30% by weight Sodium Lauroyl Methylaminopropionate-containing fluid (Kawaken Fine Chemicals))

The results showed that there was little increase in the foam volumes of Test compositions 1 and 2 comprising the larger amount of an acylamino acid type surfactant having an aspartate moiety or a glutamate moiety than that of the control composition. In contrast, Test compositions 3 to 5 which further comprised a different type of anionic surfactant showed a large increase in the foam volume and the improved quick-foaming property.

### Test Example 5-1

### Formation of Coacervate

On the basis of the results of Test examples 1 to 4, the influence in the coacervate formation of each composition was evaluated about the addition of a cationic polymer.

Washing/cleaning liquids 1 to 4 which comprised different amounts of cationic cellulose 1 which is a cationic polymer were prepared as showed in Table 5 (the amounts in the table are represented by a percentage by weight). Each of Washing liquids 1 to 4 was diluted with purified water, and then transmissions (%) of each of the resulting dilutions were measured with an ultraviolet-visible spectrophotometer (UV-1800 (Shimadzu corporation); 420 nm) at 40° C. The turbidity (%) was calculated by the following equation using the obtained transmissions, and the turbidity was used as an index of the amount of the coacervate formation to evaluate the coacervate formation.

Turbidity (%)=100−Transmission (%)

TABLE 5

| | Washing | Washing | Washing | Washing |
|---|---|---|---|---|
| | Liquid 1 | Liquid 2 | Liquid 3 | Liquid 4 |
| Sodium Lauroyl | 8.5 | 8.5 | 8.5 | 8.5 |
| Aspartate | | | | |
| Lauramidopropyl Betaine | 4.5 | 4.5 | 4.5 | 4.5 |
| Sodium Tetradecene- | 2 | 2 | 2 | 2 |
| sulfonate | | | | |
| POE Glyceryl | 2 | 2 | 2 | 2 |
| Monococoate | | | | |
| Cationic Cellulose 1 *1 | 0.1 | 0.5 | 2 | 3 |
| Solvent• | Suitable | Suitable | Suitable | Suitable |
| Preservative Agent• | Amount | Amount | Amount | Amount |
| pH Adjuster | | | | |
| Purified Water | Balance | Balance | Balance | Balance |
| Total | 100 | 100 | 100 | 100 |

*1: MERQUAT 10 (Nalco Japan)

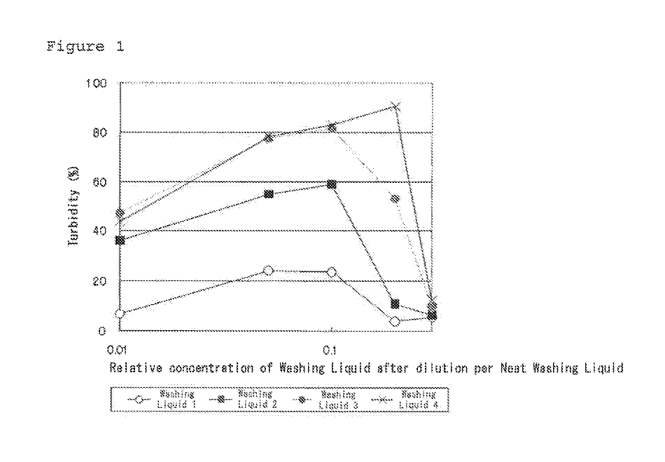

As showed in FIG. 1, there was little increase in the turbidity of the dilutions of Washing liquid 1, and the amount of a coacervate formation was small. In contrast, there were sufficient increases in the turbidity of the dilutions of Washing liquids 2 to 4. This shows that Washing liquids 2 to 4 may form the coacervate in cleaning or in rinsing away, and thereby they may provide an excellent conditioning benefit when applied to head hair as a shampoo.

### Test Example 5-2

Additionally, in order to evaluate the coacervate formation in relation to the difference of polymerization degrees of cationic cellulose, and in relation to adding other cationic polymers, Washing liquids 5 to 9 were prepared as shown in Table 6, and the change in the amount of the coacervate formation was evaluated by measuring the turbidity change in a manner similar to Test example 5-1 (the amounts of in the table are represented by a percentage by weight).

TABLE 6

| | Washing | Washing | Washing | Washing | Washing |
|---|---|---|---|---|---|
| | Liquid 5 | Liquid 6 | Liquid 7 | Liquid 8 | Liquid 9 |
| Sodium Lauroyl Aspartate | 8.5 | 8.5 | 8.5 | 8.5 | 8.5 |
| Lauramidopropyl Betaine | 4.5 | 4.5 | 4.5 | 4.5 | 4.5 |
| Sodium Tetradecenesulfonate | 2 | 2 | 2 | 2 | 2 |
| POE Glyceryl Monococoate | 2 | 2 | 2 | 2 | 2 |
| Cationic Cellulose 1 | 0.1 | 0.4 | 0.7 | 1.4 | 2.1 |
| Cationic Cellulose 2 *2 | 0.0 | 0.2 | 0.3 | 0.6 | 0.9 |
| Cationic Polymer *3 | 2 | 2 | 2 | 2 | 2 |
| pH Adjuster, | Suitable | Suitable | Suitable | Suitable | Suitable |
| Preservative Agent, | Amount | Amount | Amount | Amount | Amount |
| Solvent | | | | | |
| Purified Water | Balance | Balance | Balance | Balance | Balance |
| Total | 100 | 100 | 100 | 100 | 100 |

*2: CELQUAT SC-230M (Akzo Nobel)
*3: MERQUAT 550PR (Nalco Japan)

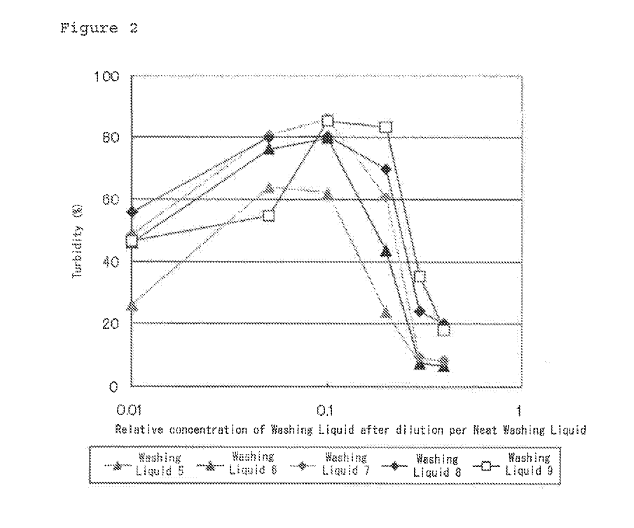

As showed in FIG. 2, Washing liquids 5 to 8 comprising cationic celluloses which have different polymerization degrees and/or another cationic polymer also showed high turbidity. On the other hand, the coacervate formation was confirmed in Washing liquid 9, but the dilution of Washing liquid 9 was heterogeneous due to clumping.

Therefore, it was shown that the coacervate formation of the composition of the present invention was improved by adding cationic cellulose or a cationic polymer.

### Test Example 6

In order to evaluate the actual effect of the composition of the present invention on hair, the following test was conducted.

A bundle of human hair was bleached and permed, and then ultra-sonicated to give a damaged hair. Washing liquid 10 (0.2 g) as showed in Table 7 (the amounts in the table are represented by a percentage by weight) was applied to the damaged hair, and then the hair was lathered up for 1 minute, and the Washing liquid was removed.

Washing liquid 10 (0.1 g) was again applied to the hair uniformly, and the hair was lathered up for 1 minute, and then the washing liquid was removed, and the hair was dried. In order to evaluate the change in the smoothness of hair after the above treatment, the difference of frictions between before and after the treatment was measured with Friction Tester KES-SE-DC (KATO TECH).

The friction tester is a device used to assess the feeling in touching an object, such as smoothness and rough texture. The rough texture is quantified as a variation of mean friction coefficient (MMD). The lower MMD is, the smoother the surface of the object is.

TABLE 7
Washing Liquid 10

| | |
|---|---|
| Sodium Lauroyl Aspartate | 8.5 |
| Lauramidopropyl Betaine | 4.5 |
| POE Glyceryl Monococoate | 2 |
| Sodium Tetradecenesulfonate | 2.1 |
| Cationic Cellulose | 1 |
| Cationic Polymer | 2 |
| Bactericidal Agent• | Suitable Amount |
| preservative Agent• | |
| Anti-Inflammatory Agent• | |
| Moisturizing Agent | |
| Solvent• | Suitable Amount |
| pH Adjuster• | |
| Flavor | |
| Algefacient | 0.3 |
| Purified Water | Balance |
| Total | 100 |

TABLE 8

| | | Hair |
|---|---|---|
| | Damaged | bundle |
| | hair | after the |
| | bundle | treatment |
| MMD of the hair bundle after the treatment | 100 | 56 |
| is shown, as compared to MMD of the | | |
| damaged hair bundle without the treatment | | |
| which is taken as 100%. | | |

As showed in Table 8, the hair bundle after the treatment with the washing liquid became smoother than the damaged hair bundle without the treatment.

This result showed that the damaged hair became smoother by the treatment with Washing liquid 10. It is thought that the treatment with Washing liquid 10 leads to a coacervate formation on hair, and thereby the texture of damaged hair is improved.

### Test Example 7

In order to evaluate the effect of the composition of the present invention on a hair surface, the structural change of a treated hair was observed.

A bundle of human hair was treated with a commercially available decoloring agent and a commercially available hairdye to give a damaged hair as a sample before treatment. The damaged hair was soaked in 10% Washing liquid 10 at 50° C. for 1 minute, and rinsed, and then dried to give a sample after treatment. The samples before and after treatment were observed with a scanning electron microscope (JSM-6380LV(JEOL Ltd.)) to evaluate the change of the surface of hair before and after treatment.

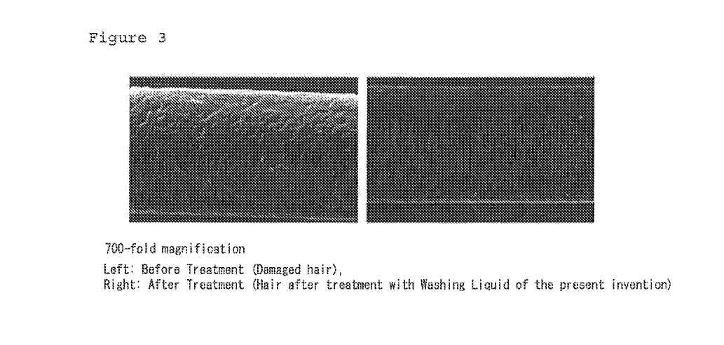

As showed in FIG. 3, the damaged hair before treatment had many damaged areas in the hair surface. In contrast, the damaged areas were reduced in the sample after treatment, and it was confirmed by such structural observation that the surface of hair became smoother.

The following formulation for cleaning scalp/head hair which has a high transparency may be prepared.

TABLE 9
Formulation example

| | |
|---|---|
| Triethanolamine-cocoyl glutamate | 2.4(% by weight) |
| Sodium Lauroyl Aspartate | 7.5 |
| Sodium Methyl Caproyl Taurate | 3.6 |
| Sodium Olefin Sulfonate (Sodium Tetradecene- | 2.1 |
| sulfonate) | |
| Alkyl Carboxymethyl Hydroxyethyl Imidazolinium | 4.5 |
| Betaine | |
| Lauryl Glycol Hydroxypropyl Ether | 3 |
| Dipropylene Glycol | 2 |
| [Hydroxy(Trimethylammonio)Propyl]Hydroxy- | 1 |
| ethylcellulose Chloride | |
| Dimethyldiallyl Ammonium Chloride•Acrylamide | 0.2 |
| Copolymer | |
| Preservative Agent•pH Adjuster | Suitable Amount |
| Purified Water | Balance |
| Total | 100 |
| (viscosity) | 2640 mPa · s |

1. A cleaning composition which comprises
Ingredient (a): an acylamino acid type surfactant 5 to 20% by weight;
Ingredient (b): a betaine-type ampholytic surfactant; and
Ingredient (c): a non-ionic surfactant 1.5 to 10% by weight,
which has a viscosity ranging from 300 to 4000 mPa·s at 20° C.

2. The cleaning composition according to claim 1 wherein the acylamino acid type surfactant of Ingredient (a) is a compound represented by the formula:
R—NH—CH(COOX₁)((CH₂)ₙCOOX₂)
wherein
R is an acyl group having 8 to 20 carbon atoms;
n is 1 or 2;
X₁ and X₂ are each independently a hydrogen atom, an alkali metal, an alkaline earth metal, an ammonium group, or a cationic residue of a basic amino acid or an alkanolamine.

3. The cleaning composition according to claim 2, wherein R is selected from the group consisting of a capryloyl group, a caprinoyl group, a lauroyl group, a myristoyl group, a palmitoyl group, a stearoyl group, an oleoyl group, a cocoyl group, an acyl group derived from palm kernel oil fatty acid, an acyl group derived from hydrogenated tallow fatty acid.

4. The cleaning composition according to any one of claims 1-3 wherein the acylamino acid type surfactant of Ingredient (a) is an anionic surfactant selected from the group consisting of lauroyl aspartic acid, cocoyl glutamic acid, and a salt thereof.

5. The cleaning composition according to any one of claims 1-4 which comprises 7 to 15% by weight of the acylamino acid type surfactant of Ingredient (a), and 2 to 5% by weight of the non-ionic surfactant of Ingredient (c).

6. The cleaning composition according to any one of claims 1-5 wherein the amount of Ingredient (b) is from 0.5 to 7 times by weight as much as the amount of Ingredient (c).

7. The cleaning composition according to any one of claims 1-6 which further comprises Ingredient (d): a cationic polymer, wherein the amount of Ingredient (d) is from 0.025 to 1 times by weight as much as the amount of Ingredient (a).

8. The cleaning composition according to claim 7 wherein the cationic polymer of Ingredient (d) comprises cationic cellulose and/or dimethyldiallyl ammonium chloride.acrylamide copolymer.

9. The cleaning composition according to any one of claims 1-8 which further comprises
Ingredient (e): an anionic surfactant other than Ingredient (a) 0.1 to 5% by weight.

10. The cleaning composition according to claim 9 wherein the anionic surfactant of Ingredient (e) is selected from the group consisting of:
an anionic surfactant which has an acyl group having 6 to 20 carbon atoms and a taurine skeleton;
an anionic surfactant which has an acyl group having 6 to 20 carbon atoms and an alanine skeleton; and
an anionic surfactant which is a sulfonic acid compound having an alkyl group and/or an alkenyl group each of which has 10 to 20 carbon atoms, or a salt thereof.

11. The cleaning composition according to any one of claims 1-10 which is applied to scalp and head hair.

12. A composition for cleaning scalp and head hair which comprises
Ingredient (a): an acylamino acid type anionic surfactant which has an aspartate moiety or a glutamate moiety 5 to 20% by weight;
Ingredient (b): a betaine-type ampholytic surfactant;
Ingredient (c): a non-ionic surfactant; and
Ingredient (d): a cationic polymer,
wherein
the amount of Ingredient (b) is from 0.25 to 2 times by weight as much as the amount of Ingredient (a);
the amount of Ingredient (c) is from 0.1 to 1 times by weight as much as the amount of Ingredient (a); and
the amount of Ingredient (d) is from 0.025 to 1 times by weight as much as the amount of Ingredient (a).

13. The composition for cleaning scalp and head hair according to claim 12 wherein a coacervate is formed when the composition is diluted by 5 to 100-fold with water.

14. The composition for cleaning scalp and head hair according to claim 12 or 13 which further comprises
Ingredient (e): an anionic surfactant other than Ingredient (a).

15. A cleaning composition which comprises
Ingredient (a): an acylamino acid type anionic surfactant which has an aspartate moiety or a glutamate moiety 5 to 20% by weight;
Ingredient (b): a betaine-type ampholytic surfactant;
Ingredient (c): a non-ionic surfactant; and
Ingredient (e): an anionic surfactant which is selected from the group consisting of:
an anionic surfactant which has an acyl group having 6 to 20 carbon atoms and a taurine skeleton;
an anionic surfactant which has an acyl group having 6 to 20 carbon atoms and an alanine skeleton; and
an anionic surfactant which is a sulfonic acid compound having an alkyl group and/or an alkenyl group each of which has 10 to 20 carbon atoms, or a salt thereof, wherein
the amount of Ingredient (b) is from 0.25 to 2 times by weight as much as the amount of Ingredient (a);
the amount of Ingredient (c) is from 0.1 to 1 times by weight as much as the amount of Ingredient (a); and
the amount of Ingredient (e) is from 0.01 to 0.6 times by weight as much as the amount of Ingredient (a).
